(19) United States
(12) Patent Application Publication
(10) Pub. No.: US 2023/0277473 A1
(43) Pub. Date: Sep. 7, 2023

(54) A Nanoparticle Composition For Allowing Sustained-Delivery And Brain-Targeting Of Risperidone And Preparation Process Thereof

(71) Applicants: Fahad A. Al-Abbasi, Jeddah (SA); Shareefa A. AlGhamdi, Jeddah (SA); Salman Bakr Hosawi, Jeddah (SA); Amira M. Alghamdi, (US); Mustafa Zeyadi, Jeddah (SA); Madan Mohan Gupta, St. Augustine (IT); Gaurav Gupta, Jagatpura (IN); Imran Kazmi, Jeddah (SA)

(72) Inventors: Fahad A. Al-Abbasi, Jeddah (SA); Shareefa A. AlGhamdi, Jeddah (SA); Salman Bakr Hosawi, Jeddah (SA); Amira M. Alghamdi, (US); Mustafa Zeyadi, Jeddah (SA); Madan Mohan Gupta, St. Augustine (IT); Gaurav Gupta, Jagatpura (IN); Imran Kazmi, Jeddah (SA)

(21) Appl. No.: 18/307,382

(22) Filed: Apr. 26, 2023

(51) Int. Cl. A61K 9/51; A23L 33/00; A61K 31/131; A61K 31/40

(52) U.S. Cl. CPC A61K 9/5153; A23L 33/30; A61K 31/131; A61K 31/40; A61K 9/5192; A61K 9/5115

(57) ABSTRACT

The present invention generally relates to a process for preparing polymeric-based nanoparticle of RPD coated with Tf and RVG comprises dissolving 1-12 wt % of RPD and 8-96 wt % of lipid in isopropyl alcohol (IPA) and heating the solution to 70° C. to create the organic phase; adding prepared organic solution to the 0-2 wt % of aqueous surfactant solution using a syringe at 70° C. temperature; swirling the obtained solution at 1000 rpm on a high speed homogenizer for 15 minutes after the solvent is evaporated using a magnetic stirrer to create the SLNs dispersion; and adding 45-100 wt % of Ethyl-3-(3-dimethylaminopropyl)carbodiimide (EDC) and 45-100 wt % of N-Hydroxysulfosuccinimide (NHS), which included activating the carboxylic acid terminal groups and conjugating Tf and RVG to RPD's PLGA nanoparticles.

### FIELD OF THE INVENTION

The present disclosure relates to a polymeric-based nanoparticle composition to increase the brain penetrance and extend the duration of action of reduced protein diet (RPD), and process of preparation thereof.

### BACKGROUND OF THE INVENTION

The effective transport of medications to the brain is one of the most urgent challenges for treating neurological illnesses. Drug effectiveness at lower doses is maximized when it is targeted towards the brain, while adverse effects are minimized by restricting drug transport to non-relevant organs. Because of the blood-brain barrier, the brain is one of the least accessible organs for medication delivery (BBB).

Atypical antipsychotic drug risperidone has been employed in the management of psychotic illnesses. Since it has less extrapyramidal side effects (EPS) than traditional antipsychotics, it has been licensed as an atypical antipsychotic drug by the U.S. Food and Drug Administration (FDA). Nevertheless, the dosage of risperidone affects EPS. Schizophrenia must be treated over an extended period of time, and the psychotic symptoms must be controlled using a low-dose RPD medication. To reduce the danger of serious side effects, it is advised to use this medication in the smallest effective dosage feasible. The medication has a substantial “first-pass” metabolism and is almost insoluble in water; its oral bioavailability is 70% (coefficient of variation: 25%). RPD and its active metabolite 9-hydroxyrisperidone have half-lives of 3 and 21 hours, respectively.

However, the fact that RPD 1) does not readily passively cross the BBB upon exogenous delivery and 2) has a short half-life of less than 3 hours presents a significant barrier to its development as a CNS medicine. However, the delivery of these compounds requires invasive surgical cannulation process. As an alternative, researchers have sought to use BBB processes to transport substances to the brain without physically or surgically disrupting the barrier.

Endogenous active transport mechanisms, such as receptor-mediated transport, are an appealing method for delivering medications to specific areas of the brain (RMT). RMT is a kind of transport mechanism that carries insulin and other macromolecules from the blood to the brain. Although transferrin receptors are known to be expressed on the luminal membrane of capillary endothelium of the BBB, transferrin (Tf) has been extensively researched and demonstrated to be a potential molecular probe for targeted drug delivery to the brain by RMT. Endothelial, intestinal, hepatocyte, and monocyte cells are the main cell types that express the TfR. As the endothelium of the brain capillaries is overexpressed with Tf-receptors (TfR), Tf-conjugated drug delivery systems (nanoparticles, liposomes, and micelles) can enhance drug transport across the BBB. As a method of delivering medication formulations to the brain, the Transferrin (Tf) system has been thoroughly investigated in earlier investigations. Tf may be adsorbed on or conjugated to a variety of polymers, such as polyethylene glycol, PLGA, lipopolyplexes, cyclodextrin derived from polymers, and gold nanoparticles. Rabies Virus Glycoprotein (RVG), a novel brain-targeting ligand that may bind to nicotinic cholinergic receptors on the BBB and promote drug formulation absorption, has also been identified in recent investigations.

Our target medicine can be packaged using nanotechnology, such as nanoparticles, and coated with Tf or RVG to exploit receptor-mediated transport to cross the BBB. For prolonged medication delivery and brain targeting, previous research has employed a range of techniques. In addition, they demonstrated that transferrin conjugated nanoparticles were more effective than unconjugated nanoparticles in reaching the brain.

In one prior art solution, a nanoparticulate system with transferrin conjugated on its surface as a molecular probe to deliver therapeutic Taxotere across the BBB. Several polymers, such as PLGA, BSA, polyethylene glycol, lipopolyplexes, polymer-based cyclodextrin, and gold nanoparticles, can encapsulate molecules. Moreover, smaller nanoparticles (less than 100 nm) could improve our formulation's ability to penetrate the BBB and enter the brain since size affects brain penetrance. Because PLGA is biocompatible, biodegradable, and has received approval from the US Food and Drug Administration, we concentrated our efforts on employing it for our nanoparticle polymers (FDA). Moreover, the RPD's half-life can be extended because to the PLGA nanoparticles' sustained release capabilities, which can lower administration frequency and boost patient compliance.

Psychotic illnesses have been treated using RPD, an atypical antipsychotic medication. Since it involves less EPS than traditional antipsychotics, the U.S. Food and Drug Administration (FDA) has authorised it as an atypical antipsychotic drug. Nevertheless, EPS are reliant on RPD dosage. To manage the psychotic symptoms, low-dose RPD therapy is necessary, and schizophrenia must be treated over an extended period of time. To reduce the danger of serious side effects, it is advised to use this medication in the smallest effective dosage feasible. RPD and its active metabolite, 9-hydroxy risperidone, have half-lives of 3 and 21 hours, respectively. The BBB limits the brain transport of RPD, which reduces the quantity of medication that reaches the target region. Moreover, a rapid release of the medication from the dosage form to the target region may lead to more adverse effects. RPD is available in US market as film coated tablet (RISPERDAL), orally dis-integration tablets (RISPERDAL), oral solution (RISPERIDONE) and as intramuscular injection (RYKINDO). All the existing formulation of RPD do not provide effective targeting and sustained delivery to brain.

According to U.S. Pat. No. 4,804,663, RPD can be created by condensation of the following two intermediates: Compound I is composed of 3- and 6-fluoro-3-(4-piperidinyl)-1,2-benzisoxazoles (2-chloroethyl)-6,7,8,9-tetrahydro-2-methyl-4H-pyrido 1,2-apyrimidin-4-one (Compound II) using catalytic amounts of potassium iodide in dimethyl formamide under basic conditions (Na2CO or KCO) (KI). At a yield of 46% overall, the crude RPD product (III) crystallizes from a mixture of DMF and isopropanol.

A raspberry and gooseberry essence, tartaric acid, na-saccharin, methyl paraben, propyl paraben, RPD, and the polyhydric alcohols sorbitol and glycerol are all disclosed in the oral solution described in Patent EP-0,196,132 (1,2,3-propanetriol). Nonetheless, it was discovered that the disclosed solutions exhibited an inadequate physicochemical stability. Surprisingly, it was discovered that sorbitol caused risperidone to break down when the solution was stored at high temperatures, simulating long-term storage conditions. The sorbitol component of the composition was removed to produce a physiochemically stable RPD solution.

The U.S. Pat. No. 5,453,425 describes an invention of an aqueous solution for oral administration that consists of water, RPD or a salt of it that is acceptable for pharmaceutical use, a buffer to keep the pH between 2 and 6, and a preservative. This solution is distinguished by the fact that it is essentially free of sorbitol, improving stability.

RPD is produced in film tablet form in Patent WO 2013/100876, which dissolves quickly and uniformly in the body, resulting in an effective therapy because of the drug's high absorption and bioavailability. The film-coated tablets were made of at least two separate diluents, the first of which was chosen from cellulose-based excipients, and the second of which was chosen from monosaccharides or disaccharides.

Patent CN1137756, described an RPD sustained release microsphere composition made employing a polymer matrix material with a molecular weight of 100,000 to 300,000. In August 2002, the long-acting antipsychotic medication Risperidal Consta (Chinese name: HENGDE), which was created based on the technology in CN1137756, hit the market. The medication is made by encapsulating RPD in a lactide-glycolide copolymer (PLGA) with a molecular weight of 150,000, suspending it in a solution, and injecting it intramuscularly once every two weeks.

In U.S. patent Ser. No. 10/098,882 B2 the active component is chosen from risperidone or one of its salts, as well as 9-hydroxy risperidone or one of its salts, in the pharmaceutical microsphere composition described in the current disclosure. The composition also contains an uncapped poly(lactide-co-glycolide). The pharmaceutical composition's weight content for the active component ranges from 10% to 60%, and its weight content for the uncapped poly(lactide-co-glycolide) does too, from 40% to 90%.

All the prior arts disclose the conventional dosage forms embodiments and they have serious limitations, such as variable absorption, considerable inter inter-individual variation in peak brain concentrations and higher systemic side effects. In addition, prior art discloses the embodiments of RPD which did not overcome BBB and provide targeted sustained delivery of RPD to brain.

In the view of the forgoing discussion, it is clearly portrayed that there is a need to have a process for formulating nanoparticle of RPD which is further adsorbed with Tf and RVG to provide effective targeted brain delivery of RPD with sustained effect.

### SUMMARY OF THE INVENTION

The present disclosure seeks to provide a polymeric-based nanoparticle composition to increase brain penetrance and extend duration of action of reduced protein diet (RPD) and a process for the same.

In an embodiment, a polymeric-based nanoparticle composition to increase brain penetrance and extend duration of action of reduced protein diet (RPD) is disclosed. The composition includes 1-12 wt % of reduced protein diet (RPD); 8-96 wt % of Compritol 888 ATO; 0-2 wt % of Poloxamer 407; 45-100 wt % of Ethyl-3-(3-dimethylaminopropyl)carbodiimide (EDC); 45-100 wt % of N-Hydroxysulfosuccinimide (NHS); 2-20 wt % of PBS; and 2-20 wt % of Rabies Virus Glycoprotein (RVG).

In an embodiment, a process for preparing polymeric-based nanoparticle of RPD coated with Tf and RVG is disclosed. The process includes dissolving 1-12 wt % of RPD and 8-96 wt % of lipid in isopropyl alcohol (IPA) and heating the solution to 70° C. to create the organic phase.

The process further includes adding prepared organic solution to the 0-2 wt % of aqueous surfactant solution using a syringe at 70° C. temperature.

The process further includes swirling the obtained solution at 1000 rpm on a high speed homogenizer for 15 minutes after the solvent is evaporated using a magnetic stirrer to create the SLNs dispersion.

The process further includes adding 45-100 wt % of Ethyl-3-(3-dimethylaminopropyl)carbodiimide (EDC) and 45-100 wt % of N-Hydroxysulfosuccinimide (NHS), which included activating the carboxylic acid terminal groups and conjugating Tf and RVG to RPD's PLGA nanoparticles.

An object of the present disclosure is to development and evaluate of novel nanoparticle formulation of RPD coated with Tf and RVG.

Another object of the present disclosure is to determine immunogenicity and cytotoxicity of development formulation.

Yet another object of the present invention is to deliver an expeditious and cost-effective process for preparing polymeric-based nanoparticle of RPD coated with Tf and RVG.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a flow chart of a process for preparing polymeric-based nanoparticle of RPD coated with Tf and RVG in accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

In an embodiment, a polymeric-based nanoparticle composition to increase brain penetrance and extend duration of action of reduced protein diet (RPD) is disclosed. The composition includes 1-12 wt % of reduced protein diet (RPD); 8-96 wt % of Compritol 888 ATO; 0-2 wt % of Poloxamer 407; 45-100 wt % of Ethyl-3-(3-dimethylaminopropyl)carbodiimide (EDC); 45-100 wt % of N-Hydroxysulfosuccinimide (NHS); 2-20 wt % of PBS; and 2-20 wt % of Rabies Virus Glycoprotein (RVG).

In another embodiment, weight percentage of the Poloxamer 407, Ethyl-3-(3-dimethylaminopropyl)carbodiimide (EDC), and N-Hydroxysulfosuccinimide (NHS) is preferably 2%, 98%, and 98%, respectively.

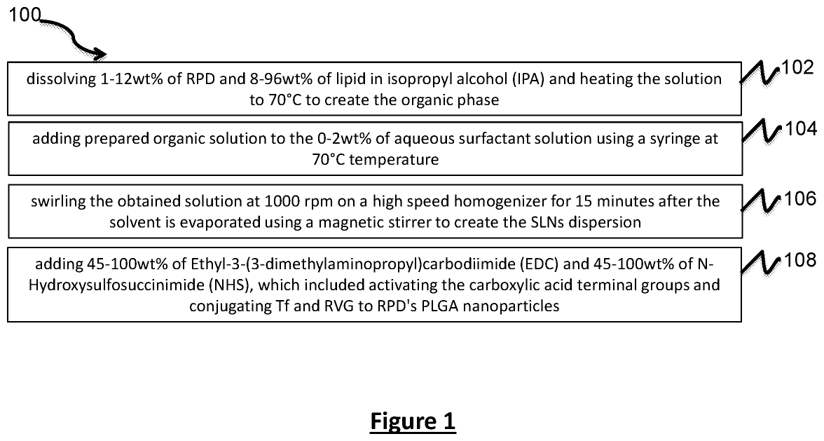

Referring to FIG. 1, a flow chart of a process for preparing polymeric-based nanoparticle of RPD coated with Tf and RVG is illustrated in accordance with an embodiment of the present disclosure. At step 102, process 100 includes dissolving 1-12 wt % of RPD and 8-96 wt % of lipid in isopropyl alcohol (IPA) and heating the solution to 70° C. to create the organic phase.

At step 104, process 100 includes adding prepared organic solution to the 0-2 wt % of aqueous surfactant solution using a syringe at 70° C. temperature.

At step 106, process 100 includes swirling the obtained solution at 1000 rpm on a high-speed homogenizer for 15 minutes after the solvent is evaporated using a magnetic stirrer to create the SLNs dispersion.

At step 108, process 100 includes adding 45-100 wt % of Ethyl-3-(3-dimethylaminopropyl)carbodiimide (EDC) and 45-100 wt % of N-Hydroxysulfosuccinimide (NHS), which included activating the carboxylic acid terminal groups and conjugating Tf and RVG to RPD's PLGA nanoparticles.

In one embodiment, the RPD-NPs are prepared by solvent diffusion-solvent evaporation method using Compritol 888 ATO as the lipid and Poloxamer 407 as the surfactant.

In one embodiment, the evaporated solvent is cooled by ice bath with continuous stirring at 1000 rpm on high speed homogenizer for 15 min to form SLNs dispersion.

In one embodiment, the process further comprises adding 2 ml of EDC is 2 mg/ml in water and 2 ml NHS in 2 mg/ml in water and further adding 20 ml of nanoparticle suspension containing 80 mg of RPD-loaded PLGA nanoparticles.

In one embodiment, Carboxylic acid groups at the periphery are converted to amine-reactive esters by stirring the PLGA with EDC and Sulfo-NHS reaction mixture at room temperature for 4 h.

In one embodiment, activated NPs are dispersed in 1 ml of PBS, and 1 ml, 1 mg/ml of Tf or 1 ml, 1 mg/ml of RVG is added drop-wise to the mixture.

In one embodiment, the mixture is stirred at room temperature for 2 h and incubated at 4° C. overnight or 12 h, wherein the samples are washed and lyophilized using a freeze dryer.

In one embodiment, the RPD and lipid is preferably dissolved in isopropyl alcohol (IPA)1:8 ratio.

Development of Nanoparticle Formulation of RPD Coated with Tf and RVG

i) Development of RPD Nanoparticle (RPD-Np) Formulation

RPD-Np are created utilizing the solvent diffusion-solvent evaporation process using Poloxamer 407 as the surfactant and Compritol 888 ATO as the lipid. RPD and lipid (drug:lipid ratio 1:8) are dissolved in isopropyl alcohol (IPA) and heated to 70° C. to create the organic phase. At the same temperature and using a syringe, organic solution is added to aqueous surfactant solution (2% v/v). To create the SLNs dispersion, it is continuously swirled at 1000 rpm on a high speed homogenizer for 15 minutes after the solvent had been evaporated using a magnetic stirrer.

ii) Tf and RVG Conjugation to Peptide-Loaded Nanoparticles

Two steps are used to conjugate Tf and RVG to RPD's PLGA nanoparticles. Ethyl-3-(3-dimethylaminopropyl)carbodiimide (EDC) and N-Hydroxysulfosuccinimide (NHS) (98%) are needed for the first step, which included activating the carboxylic acid terminal groups. Afterwards, the nanoparticles are conjugated with Tf or RVG.
In a nutshell, 20 ml of a nanoparticle solution comprising 80 mg of RPD-loaded PLGA nanoparticles is mixed to 2 ml of EDC and NHS (2 ml, 2 mg/ml in water). The PLGA with EDC and Sulfo-NHS reaction mixture is stirred at room temperature for 4 hours to transform carboxylic acid groups at the periphery into amine-reactive esters.
Thereafter, 1 ml of PBS containing the activated RPD-Np is added, followed by dropwise additions of Tf (1 ml, 1 mg/ml) or RVG (1 ml, 1 mg/ml). The mixture is mixed for two hours at room temperature before being incubated for 12 hours at 4° C. Next, a freeze dryer is used to lyophilize the samples after they had been cleaned.

Evaluation of RPD Nanoparticles Conjugated with Tf or RVG

Drug-excipients compatibility Study: Compatibility studies are carried out to find out whether the polymeric excipients used in formulation are compatible with drug throughout shelf life. These studies have been performed by using differential scanning, using aluminum hermetic pans with pierced lid over range of 30° C. to 300° C., at a scan rate of 10° C./min with nitrogen at the flow of 50 mL/min as a purge gas. The Fourier transform infrared spectroscopy using KBr pellets are conducted at a scan range of 450-4000 cm-1.

Particle size and zeta potential determination: The particle size and zeta potential of SLN's are determined by using the Zeta sizer. Sample quantity of 1 ml is taken and diluted to 10 ml with distilled water. The size determination is carried out in samples after 10 minutes of sonication

Surface morphology: The shape of prepared SLN's is determined by using SEM. A small sample quantity is kept on surface of small metal stubs by help of adhesive tape. The sample coating is done with gold by help of sputter coater before testing. The SEM analysis is done by applying fixed voltages.

Determination of entrapment efficiency (EE): The EE of developed RPD-Np is determined by ultra-sonicating the RPD-Np dispersion at 20000 rpm for 30 minutes. The supernatant obtain is diluted with 10 ml ethanol and drug concentration is determined spectrophotometrically (Shimadzu 1800, Japan) at 240 nm. The EE percentage is calculated by using below formula.

EE (%)=[(W_{initial drug}−W_{free drug})/W_{initial drug}]×100

Where ‘W_{initial drug}’ is the initial drug weight taken for the formulation and W_{free drug} free drug is the weight of free drug obtain in supernatant solution after sonication.

In-vitro drug release study: The in-vitro drug release from RPD-SLNP's is performed by using technique of dialysis. The dialysis membrane with molecular weight cut off 10,000 to 12,000 Da is used for the study. 10 mg of RPD-Np is placed in to the dialysis bag and tied properly with help of a thread. The dialysis bags containing free drug and RPD-Np are immersed in a container having 250 ml of dissolution medium PBS:ethanol (8:2; pH 6.8). The container is previously placed on an electromagnetic stirrer maintained at 37±0.5° C. temperature and dissolution medium are continuously stirred at 100 rpm by electromagnetic bead. Sample of 5 ml is withdrawn from dissolution medium at 1, 2, 4, 6, 8-, 10-, 12- and 24-hour time intervals and replenish by the same volume of fresh dissolution medium with same temperature (37±0.5° C.). RPD concentration is determined spectrophotometrically at 250 nm.

Stability studies: Stability studies are carried out on developed RPD-Np formulation as per ICH guideline at accelerate condition (40° C./75% RH) and at long term condition (25° C./60% RH). After specified interval formulation is checked for all evaluation parameters

The developed invention has the following advantages over the existing formulations.
Present invention provides better penetration of drug through BBB due to nano size drug particles thereby provide higher bioavailability of drug
The present invention provides quick onset of action drug at target site thereby increasing effectiveness of drug.
The present invention will exhibit reduced side effects due to its longer duration of action and decreased frequency of administration.
The present invention significantly increases the patient compliance due ease of application, reduced dose frequency and long duration of action.
The present invention also sustained action of the drug to the target site.
Formulation of Nanoparticle with conjugation with Tf, RVG is more complex as compared to existing tablet and topical formulations
The evaluation of RPD-NPs is a complicated process and requires specialized instrument and technician to perform the evaluation tests.
The present invention requires use of a special ingredients like PLGA, Tf, and RVG for formulation. Hence, availability of this agent is critical factor.
The characterization of developed invention is very complex and any discrepancy could result in batch-to-batch variation.
The present invention is more susceptible to stability problem due to chemical degradation. Hence, effective and strong packing material should to be used to protect it from harmful effect of environment.

RPD-NPs are prepared by solvent diffusion-solvent evaporation method using Compritol 888 ATO as the lipid and Poloxamer 407 as the surfactant. Organic phase is prepared by dissolving RPD and lipid (drug:lipid ratio 1:8) in isopropyl alcohol (IPA) and heated up to 70° C. With the help of syringe, organic solution is added to aqueous surfactant solution (2% v/v) at the same temperature. It is stirred on magnetic stirrer to evaporate the solvent and then cooled by ice bath with continuous stirring at 1000 rpm on high-speed homogenizer for 15 min to form SLNs dispersion.

Take 2 ml (2 mg/ml in water) of EDC and NHS (2 ml, 2 mg/ml in water) is added to 20 ml of nanoparticle suspension containing 80 mg of RPD-loaded PLGA nanoparticles. Carboxylic acid groups at the periphery are converted to amine-reactive esters by stirring the PLGA with EDC and Sulfo-NHS reaction mixture at room temperature for 4 h.

The activated NPs are then dispersed in 1 ml of PBS, and Tf (1 ml, 1 mg/ml) or RVG (1 ml, 1 mg/ml) is added drop-wise to the mixture. The mixture is stirred at room temperature for 2 h and incubated at 4° C. overnight or 12 h. Finally, the samples are washed and lyophilized using a freeze dryer.

The developed process increases penetration through BBB hence providing higher bioavailability, provides longer duration of action, promotes Targeted drug delivery to Brain, provides quick onset of action, reduces frequency of drug administration, reduces adverse effect of drug due to sustained action, increases patient compliance, and promotes better and efficient treatment of brain disorders thereby reduced cost of treatment.

The developed composition is used in the field of Medicine for treatment of various skin disorders and the process is used pharmaceutical industries for manufacturing the product.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A polymeric-based nanoparticle composition to increase brain penetrance and extend duration of action of reduced protein diet (RPD), the composition comprises:
1-12 wt % of reduced protein diet (RPD);
8-96 wt % of Compritol 888 ATO;
0-2 wt % of Poloxamer 407;
45-100 wt % of Ethyl-3-(3-dimethylaminopropyl)carbodiimide (EDC);
45-100 wt % of N-Hydroxysulfosuccinimide (NHS);
2-20 wt % of PBS; and
2-20 wt % of Rabies Virus Glycoprotein (RVG).

2. The composition as claimed in claim 1, wherein weight percentage of the Poloxamer 407, Ethyl-3-(3-dimethylaminopropyl)carbodiimide (EDC), and N-Hydroxysulfosuccinimide (NHS) is preferably 2%, 98%, and 98%, respectively.

3. A process for preparing polymeric-based nanoparticle as claimed in claim 1, the composition comprises:
dissolving 1-12 wt % of RPD and 8-96 wt % of lipid in isopropyl alcohol (IPA) and heating the solution to 70° C. to create the organic phase;
adding prepared organic solution to the 0-2 wt % of aqueous surfactant solution using a syringe at 70° C. temperature;
swirling the obtained solution at 1000 rpm on a high speed homogenizer for 15 minutes after the solvent is evaporated using a magnetic stirrer to create the SLNs dispersion; and
adding 45-100 wt % of Ethyl-3-(3-dimethylaminopropyl)carbodiimide (EDC) and 45-100 wt % of N-Hydroxysulfosuccinimide (NHS), which included activating the carboxylic acid terminal groups and conjugating Tf and RVG to RPD's PLGA nanoparticles.

4. The process as claimed in claim 3, wherein the RPD-NPs are prepared by solvent diffusion-solvent evaporation method using Compritol 888 ATO as the lipid and Poloxamer 407 as the surfactant.

5. The process as claimed in claim 3, wherein the evaporated solvent is cooled by ice bath with continuous stirring at 1000 rpm on high speed homogenizer for 15 min to form SLNs dispersion.

6. The process as claimed in claim 3, further comprises adding 2 ml of EDC is 2 mg/ml in water and 2 ml NHS in 2 mg/ml in water and further adding 20 ml of nanoparticle suspension containing 80 mg of RPD-loaded PLGA nanoparticles.

7. The process as claimed in claim 6, wherein Carboxylic acid groups at the periphery are converted to amine-reactive esters by stirring the PLGA with EDC and Sulfo-NHS reaction mixture at room temperature for 4 h.

8. The process as claimed in claim 3, further comprises dispersing the activated NPs in 1 ml of PBS, and 1 ml, 1 mg/ml of Tf or 1 ml, 1 mg/ml of RVG is added drop-wise to the mixture.

9. The process as claimed in claim 8, wherein the mixture is stirred at room temperature for 2 h and incubated at 4° C. overnight or 12 h, wherein the samples are washed and lyophilized using a freeze dryer.

10. The process as claimed in claim 3, wherein the RPD and lipid is preferably dissolved in isopropyl alcohol (IPA)1:8 ratio.
